(19) United States
(12) Patent Application Publication
(10) Pub. No.: US 2009/0169524 A1
(43) Pub. Date: Jul. 2, 2009

(54) COMPOSITES AND METHODS OF PREPARATION AND USE THEREOF

(76) Inventors: Kalpana S. Katti, Fargo, ND (US); Dinesh R. Katti, Fargo, ND (US)

Correspondence Address: VIKSNINS HARRIS & PADYS PLLP, P.O. BOX 111098, ST. PAUL, MN, 55111-1098, US

(21) Appl. No.: 12/274,981

(22) Filed: Nov. 20, 2008

Related U.S. Application Data
(60) Provisional application No. 60/989,659, filed on Nov. 21, 2007.

(51) Int. Cl. A61K 35/32; A61K 33/42; A61P 19/00

(52) U.S. Cl. 424/93.7; 424/602

(57) ABSTRACT

The invention provides composites, methods for their preparation, composites prepared according to the methods, and methods for using the composites.

### RELATED APPLICATION(S)

This patent document claims the benefit of priority of U.S. application Ser. No. 60/989,659, filed Nov. 21, 2007, which application is herein incorporated by reference.

### STATEMENT OF GOVERNMENT SUPPORT

This invention was made with government support under Grant # 0132768 awarded by the National Science Foundation. The government has certain rights in the invention.

### BACKGROUND

There is a need for materials for biomedical applications, especially in the field of tissue engineering.

### SUMMARY OF CERTAIN EMBODIMENTS OF THE INVENTION

As described herein, biopolymer-based nanocomposites can be used, e.g., to replace synthetic polymer composites for various biomedical applications. This is because, e.g., many biopolymers are biocompatible and biodegradable. In the present work, a biopolymer-based nanocomposite including chitosan, montmorillonite (MMT) and hydroxyapatite (HAP) was developed. Such a composite is useful, e.g., for biomedical applications. The composite was prepared from chitosan, unmodified MMT, and HAP precipitate in aqueous media. The properties of the composites were investigated using X-ray diffraction (XRD), Fourier transform infrared spectroscopy (FTIR), atomic force microscopy (AFM), thermogravimetric analysis (TGA) and nanoindentation. The biocompatibilities of the composites were investigated by seeding samples with osteoblasts. The XRD results reveal that an intercalated structure was formed with an increase in d-spacing. FTIR studies provide evidence of molecular interaction among the three different constituents of the composite. AFM images indicate a microstructure with well distributed nanoparticles in the chitosan matrix. The composites also exhibit a significant enhancement in nanomechanical property as compared to pure chitosan. The TGA results indicate that an intercalated nanocomposite was formed with improved thermal property. Cell culture test results indicated that MMT and MMT/HAP composites are highly biocompatible. From all these results it is concluded that the composites are useful for various biomedical applications.

Accordingly, certain embodiments of the present invention provide composite materials that comprise chitosan, montmorillonite, and hydroxyapatite, useful, e.g., for bone tissue engineering.

In some embodiments of the invention, the composite can be prepared by dispersing montmorillonite, e.g., unmodified montmorillonite, in a chitosan solution and by adding hydroxyapatite precipitate to the solution.

### BRIEF DESCRIPTION OF THE FIGURES

FIG. 1. Photographs of the cells grown over MMT/HAP mix after (a) 3 days and after (b) 7 days.

FIG. 2. XRD pattern of (a) HAP (b) MMT (c) chitosan (d) Chi/HAP (e) Chi/MMT (f) Chi/MMT/HAP.

FIG. 3. Schematic diagram of possible interactions in chi/MMT/HAP composite.

FIG. 4. PA-FTIR spectra of (a) chitosan powder (b) chitosan sheet.

FIG. 5. PA-FTIR spectra of (a) HAP (b) MMT (c) chitosan (d) Chi/HAP (e) Chi/MMT (f) Chi/MMT/HAP.

FIG. 6. PA-FTIR spectra of (a) HAP (b) MMT (c) chitosan (d) Chi/HAP (e) Chi/MMT (f) Chi/MMT/HAP in the energy range 3200-4000 cm⁻¹.

FIG. 7. PA-FTIR spectra of (a) HAP (b) MMT and double derivative spectra of (c) chitosan (d) Chi/HAP (e) Chi/MMT (f) Chi/MMT/HAP in the energy range 900-1900 cm⁻¹.

FIG. 8. Thermogravimetric plots of chitosan, chi/MMT and chi/MMT/HAP.

FIG. 9. Atomic force microscope phase images of (a) chi/MMT (b) chi/HAP and (c) chi/MMT/HAP composites.

FIG. 10. Comparison of the cell growth over MMT, HAP and MMT/HAP mix.

FIG. 11. Osteoblast cell growth. Upper images are photographs showing cell growth over MMT after (a) 1 day (b) 3 days and (c) 7 days of feeding the cells. Lower images are photographs showing cell growth over 1:1 MMT/HAP mix after (a) 1 day (b) 3 days and (c) 7 days of feeding the cells.

FIG. 12. Transmission spectra of MMT and OMMT.

FIG. 13. H—O—H banding region of transmission FTIR spectra of MMT and OMMT.

FIG. 14. Si—O stretching region of transmission FTIR spectra of MMT and OMMT. Second derivative spectra of OMMT (top) and MMT (bottom) are also shown in inset.

FIG. 15. Si—O stretching region of FTIR spectra of MMT and OMMT which have been modified by three different modifiers. (a) MMT, (b) MMT modified by diaminododecane, (c) MMT modified by ndodecylamine and (d) MMT modified by aminolauric acid.

FIG. 16. XRD patterns and d (001) spacing of MMT and OMMT. d (001) spacing of OMMT has increased from 11.47 Å to 13.76 Å due to the intercalation of aminopimelic acid.

### DETAILED DESCRIPTION

Certain embodiments of the present invention provide composites comprising chitosan, montmorillonite, and hydroxyapatite.

Certain embodiments of the present invention provide methods for preparing a composite that comprises chitosan, montmorillonite, and hydroxyapatite, comprising dispersing montmorillonite in a chitosan solution and adding hydroxyapatite precipitate to the solution so as to prepare a composite that comprises chitosan, montmorillonite, and hydroxyapatite.

Certain embodiments of the present invention provide composites that comprise chitosan, montmorillonite, and hydroxyapatite prepared according to the methods described herein.

In certain embodiments, the composite further comprises osteoblast cells.

In certain embodiments, the composite further comprises calcium mineralization.

Certain embodiments of the present invention provide methods for treating a patient having a damaged bone, comprising inserting into the patient a composite as described herein so as to treat the damaged bone.

In certain embodiments, the bone was damaged by an injury.

In certain embodiments, the bone was damaged by a disease.

In certain embodiments, the damaged bone is a portion of a joint.

Certain embodiments of the present invention provide compositions comprising a composite as described herein and an acceptable carrier.

In certain embodiments, the carrier is a pharmaceutically acceptable carrier.

Certain embodiments of the present invention provide composite as described herein for use in medical treatment or diagnosis.

Certain embodiments of the present invention provide the use of a composite as described herein to prepare a medicament useful for treating a disease or injury in an animal.

In certain embodiments, the disease or injury is a disease or injury of a bone.

In certain embodiments, the bone is a portion of a joint.

In certain embodiments, the animal is a mammal.

In certain embodiments, the mammal is a human.

Certain embodiments provide composites as described herein use in medical treatment or diagnosis.

Certain embodiments provide the use of a composite as described herein to prepare a medicament useful for treating a disease or injury in an animal.

Certain embodiments provide methods and intermediates useful, e.g., for preparing composites.

Certain embodiments provide clays, such as montmorillonite, that comprise an unnatural amino acid (i.e., not one of the 20 standard amino acids), such as 2-aminopimelic acid, 5-aminovaleric acid or DL-2-aminocaprylic acid (see Table 1 and Example 2). These materials are useful, e.g., for preparing composites for bone tissue engineering. In certain embodiments, the clay is modified, e.g., with small organic molecules. In certain embodiments, the clay is modified with molecules that are biocompatible molecules. In certain embodiments, the clay is unmodified clay. In certain embodiments, the clay is modified clay, e.g., organically modified clay (see, e.g., Sikdar et al., Journal of Nanoscience and Nanotechnology, 8(4), 1638-1657 (2008); Sikdar et al., J. Appl. Pol. Sci., 107, 3137-3148 (2008); and Sikdar et al., Journal of Applied Polymer Science, 105, 790-802 (2007)).

TABLE 1

| Name | Structure (from Sigma Aldrich) | Formula | Source |
|---|---|---|---|
| 2-Aminopimelic acid | 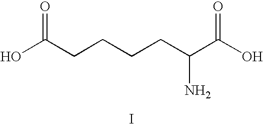 | C₇H₁₃NO₄ | Sigma Aldrich |
| 5-Aminovaleric acid | 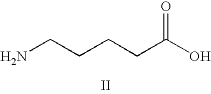 | C₅H₁₁NO₂ | Sigma Aldrich |
| DL-2- Aminocaprylic acid | 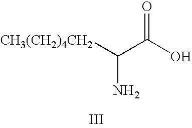 | C₈H₁₇NO₂ | Sigma Aldrich |

There is a need for the development of composite materials useful for biomedical applications, especially in the field of tissue engineering. Natural polymers may be more useful than synthetic polymers because of their typically better biocompatibility and nontoxic degradation products. Of natural polymers, chitosan-based materials have been investigated for a wide range of applications, e.g., bone substitutes, drug delivery system, food packaging, and artificial skin (Khor et al., Biomaterials, 24, 2339-2349 (2003); Suh et al., Biomaterials, 21, 707-715 (2000); Sivakumar et al. Carbohydrate Polymers, 49, 281-288 (2002); and Sébastien et al., Carbohydrate Polymers, 65, 185-193 (2006)). This is due at least in part to some unique properties of chitosan, such as bioactivity, biodegradability, biocompatibility, solubility in aqueous medium, and ability to form complexes (Qurashi et al., J. Appl. Polym. Sci., 46, 255-261 (1992); Onishi et al., Biomaterials, 20, 175-182 (1999); Vande Vord et al., J Biomed Mater Res, 59, 585-590 (1999); and Kumar, React Funct Polym, 46, 1-27 (2000)).

Chitosan is a natural polysaccharide derived from chitin, which is the second most abundant natural polymer after cellulose. Chitosan is made of glucosamine and N-acetyl glucosamine units, linked in a β (1-4) manner (Rinaudo, Prog. In Polym. Sci., 31, 603-632 (2006); Dumitriu, Polymeric biomaterials. New York: Marcel Dekker, Inc.; p 187 (2002); Martino et al., Biomaterials, 26, 5983-5990 (2005); Kas, J. Microencapsulation, 14, 689-711 (1997)) and can be obtained by partially or fully deacetylation of chitin. In many circumstances, it would be desirable to improve the solubility, thermal and physicochemical properties of chitosan.

In an effort to obtain better properties, organic and inorganic materials may be added to chitosan, for example cellulose, PLA/PGA, montmorillonite, PEG, PVA, calcium phosphates and hydroxyapatite (HAP). HAP can be useful because of its bioactivity, biocompatibility and osteoconductive properties. These composites may provide good mechanical properties, biodegradability, antibacterial activity, and hydrophilicity. Further, chitosan/calcium phosphate and chitosan/HAP composites may be material useful for tissue engineering applications. A chitosan/HAP composite scaffold can be prepared using a lyophilization method. These scaffolds have very good porosity, better biocompatibility, and have demonstrated higher proliferation of cells on it than chitosan scaffolds, and more apatite was grown on the composite scaffold than that of the pure chitosan scaffold. The bioactivity of the composites typically increased with HAP particles. Further, addition of HAP particles to chitosan reduces the water absorption, and as a result increases the mechanical property under moisture condition. Moreover, chitosan/HAP composites are biologically well tolerated and osteoconductive in nature, which is important for tissue engineering applications.

Montmorillonite (MMT), a layered alumina silicate, belongs to the smectite group of minerals. Its structure includes one edge shared octahedral sheet of aluminum hydroxide fused in between two silica tetrahedra. Nanoclay acts as a suitable filler material because of its high surface area and large aspect ratio. MMT enhances the mechanical properties and thermal stability when it is reinforced with chitosan. Addition of nanoclay to polymer significant improves mechanical and thermal properties. Polymer/layered silicate nanocomposites have been investigated for structural applications. A method was developed by Lin to prepare chitosan/MMT composites (Lin et al., J Polym Sci, 98, 2042-2047 (2005)). MMT was first modified with potassium persulfate before mixing with chitosan solution. The resulting composite has improved mechanical properties. The in vitro degradation effect was also studied and it was observed that the exfoliated MMT hindered the degradation behavior of chitosan molecule. Thus, in certain embodiments, MMT is modified, e.g., with small organic molecules. In certain embodiments, the MMT is modified with molecules that are biocompatible molecules. In certain embodiments, the montmorillonite is unmodified montmorillonite. In certain embodiments, the montmorillonite is modified montmorillonite, e.g., organically modified montmorillonite (see, e.g., Sikdar et al., Journal of Nanoscience and Nanotechnology, 8(4), 1638-1657 (2008); Sikdar et al., J. Appl. Pol. Sci., 107, 3137-3148 (2008); and Sikdar et al., Journal of Applied Polymer Science, 105, 790-802 (2007)).

There have been no reports of combining chitosan, MMT and HAP together to make a composite. As described herein, a novel nanocomposite prepared from chitosan, MMT and hydroxyapatite has been developed. The chitosan/MMT/HAP composite was carefully studied by X-ray diffraction (XRD), Fourier transform infrared spectroscopy (FTIR), Atomic force microscopy (AFM), and Thermogravimetric analysis (TGA). The mechanical properties were also evaluated using nanoindentaion technique. Biocompatibility of MMT/HAP mix was also evaluated by conducting tissue engineering experiments.

The invention will now be illustrated by the following non-limiting Examples.

### Example 1

### A Chitosan-Montmorillonite-Hydroxyapatite Composite

Described herein is the synthesis and characterization of a chitosan-montmorillonite-hydroxyapatite nanocomposite, useful, e.g., for bone tissue engineering.

For the first time, a chitosan/MMT/HAP nanocomposite was successfully prepared, and the properties of the materials were investigated. XRD results revealed that an intercalated structure was achieved with an increase in d-spacing. FTIR spectra of the composites exhibited the presence of characteristic organic and inorganic absorption bands. This study demonstrated that there was a significant interaction taking place among the three constituents of the composite, which is clearly observed from the shift in band position of chitosan in presence of MMT and HAP. It has been found from TGA results that adding the MMT and HAP inorganic particles to biopolymer chitosan has improved the thermal stability of the composites. These MMT and HAP nanoparticles are homogeneously distributed in the polymer matrix in all composites, which is seen in the AFM images. There is a significant improvement in the nanomechanical properties of the composites, which provides a material for tissue engineering applications. This notable improvement in mechanical property is attributed to the better dispersion of nanoparticles and the interaction between nanoparticles and organic matrix. From the cell culture studies, it appears that the chitosan/clay mix is biocompatible and allows for growth and proliferation of osteoblasts.

### Results

### XRD

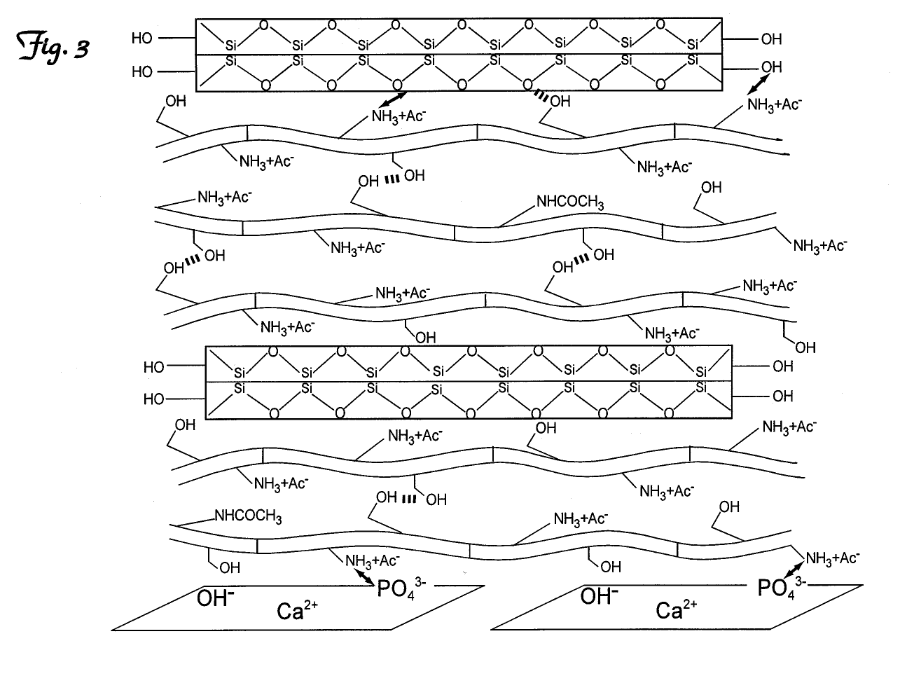

XRD patterns of chitosan, MMT, HAP and their composites are shown in FIG. 2. From the diffraction peaks, d-spacing can be determined using Bragg's equation (nλ=2d sin θ). In the figure, pure MMT shows the basal d₀₀₁ peak at 2θ=7.9, which corresponds to a d-spacing of 11.11 A°. In pure chitosan there are two broad peaks, one at 2θ=9.4° and another at 2θ=21°. A Chi/MMT composite has 10 wt % of MMT. Thus, after mixing 10 wt % of MMT with chitosan, the basal peak of MMT has shifted from 7.9 to 6.3, which changes the d-spacing from 11.11 A° to 14.01 A°. The 2.9A° increase in d-spacing indicates the formation of intercalated chi/MMT structure. In acidic media, NH₂ group of chitosan has converted to NH₃⁺. Since the polymer is hydrophilic in nature, it exhibits greater miscibility with MMT. So both the hydrophilicity and polycationic nature of chitosan appear to force the polymer molecule to go inside the MMT interlayer through cation exchange process. As a result, it strongly facilitates electrostatic interaction between chitosan and negatively charged clay surface. In a chi/MMT/HAP composite, adding HAP to Chi/MMT solution, the basal peak appears at 6.60. HAP peaks appear at 26° and 32°, which evidenced the presence of HAP molecule in the chi/MMT/HAP composite. Although the d-spacing is less than that of chi/MMT composite, it is still greater than pure MMT. HAP molecule has PO₄³⁻ groups and its surface is basically negatively charged which, can also interact with the cations of chitosan. So, when HAP is added, it is thought that the ammonium ions of chitosan also start interacting with HAP molecule. Hence, the change in d-spacing can be attributed to the interaction of chitosan molecules with both MMT and HAP. FIG. 3 shows the schematic diagram of the possible interaction among chitosan, MMT and HAP.

### FTIR

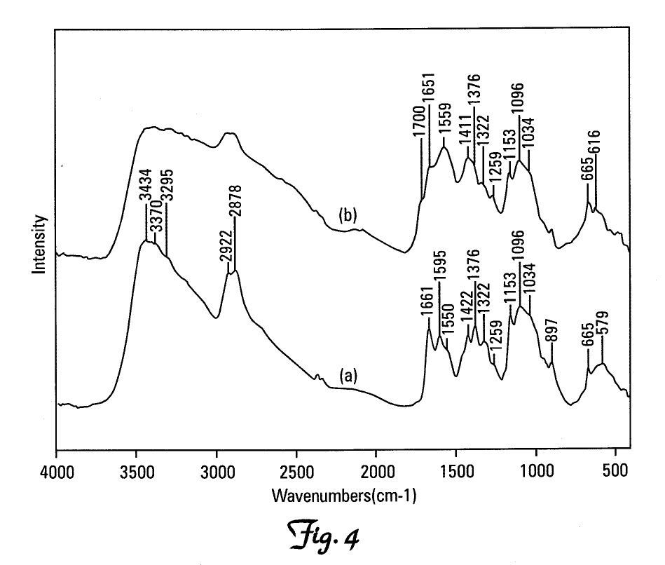

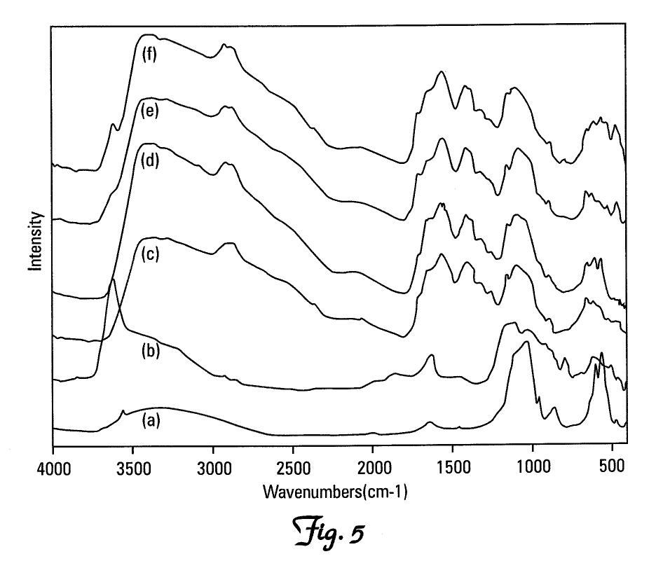

FTIR spectra of chitosan and nanocomposite sheets were studied in order to investigate the interaction between the chitosan and nanoparticles. FIG. 4 shows the photoacoustic spectra of chitosan powder and chitosan sheets. Band assignments for chitosan are given in Table 2. In powder chitosan, O—H stretching band appears at 3434 cm⁻¹ and N—H stretching bands appear at 3295 cm⁻¹, 3370 cm⁻¹. From the spectra, it can be clearly observed that after chitosan is processed into sheets, N—H stretching band became broader. It indicates the formation of ammonium ion in acidic solution and O—H broad band is also overlapping with N—H. N—H deformation band of chitosan powder has shifted from 1595 cm⁻¹ to 1559 cm⁻¹. Other O—H vibrational band positions at 1422 cm⁻¹ and 579 cm⁻¹ have also changed because of its interaction with water in solution. The FTIR spectrum of processed chitosan can be used to compare with the composites. FTIR spectra of chitosan, MMT, HAP and all the composites are shown in FIG. 5. Spectra of chitosan and its composites will be compared.

TABLE 2
Band assignments of chitosan.

| Wavenumbers (cm⁻¹) | Band assignment |
|---|---|
| 3434 | O—H stretching |
| 3370 | N—H asymmetric stretching |
| 3295 | N—H symmetric stretching |
| 2922 | C—H asymmetric stretching |
| 2878 | C—H symmetric stretching |
| 1661 | Amide I band |
| 1595 | NH2 in amino group |
| 1550 | Amide II band |
| 1422 | O—H and C—H in the ring |
| 1376 | C—H bending in amide group |
| 1322 | C—H in the ring |
| 1259 | NH2 rocking |
| 1034-1153 | C—O stretching |
| 897 | C—C—O |
| 665 | N—H out of plane deformation |
| 579 | O—H out of plane deformation |

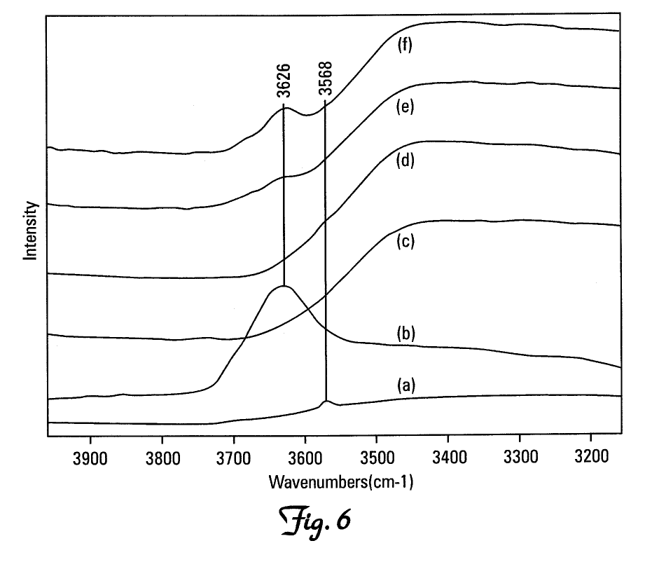

FIG. 6 presents the spectra in the energy range 3200-4000 cm⁻¹. Structural O—H stretching of MMT appears in chi/MMT and chi/MMT/HAP composites at the same position, i.e., at 3626 cm⁻¹. This gives an evidence of the presence of MMT in both the intercalated composites. Again, O—H stretching of hydroxyapatite at 3568 cm⁻¹ is present in chi/HAP and chi/MMT/HAP composites although the intensity is low.

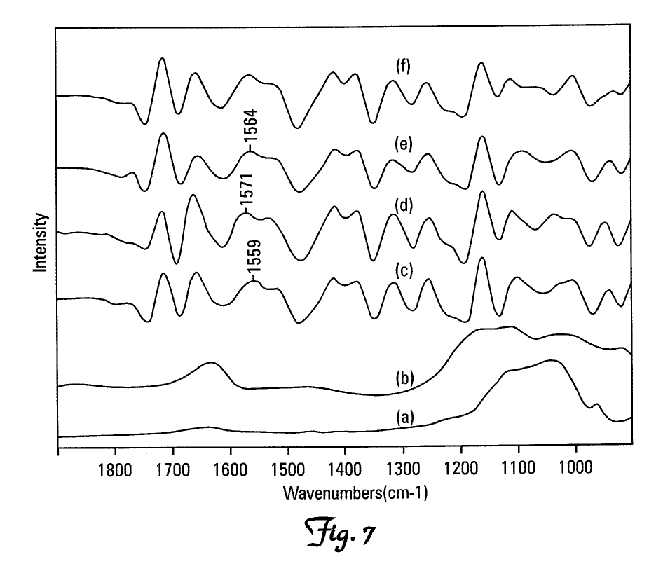

FIG. 7 shows the spectra of MMT, HAP and double derivative spectra of chitosan and its composites in the energy range 900-1900 cm⁻¹. If one carefully observes the N—H bending region, it can be noticed that N—H bending has shifted from 1559 cm⁻¹ to 1571 cm⁻¹ in chi/HAP composite and to 1564 cm⁻¹ in chi/MMT and chi/MMT/HAP composites respectively. Since these bands are compared with processed chitosan, all these changes in band position suggest some electrostatic interaction among chitosan, MMT and HAP. PO₄³⁻ bands of HAP, Si—O bands of MMT and C—O bands of chitosan appear in between 1000-1200 cm⁻¹. So the composite spectra show the combination of all the bands in this region.

### TGA

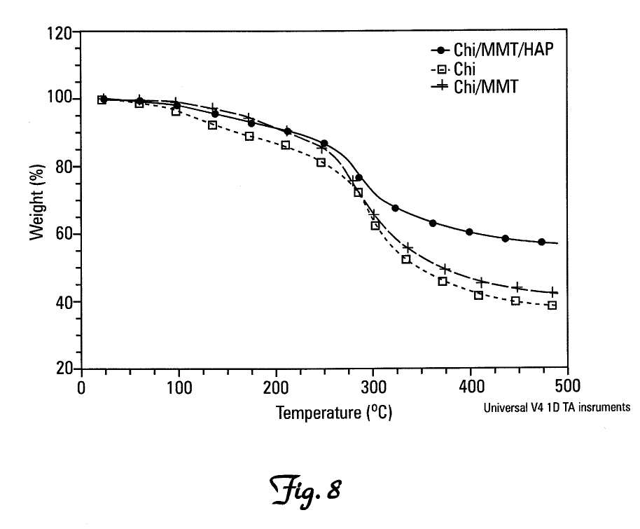

Thermogravimetric analysis was done in order to find out the thermal stability of the composites. Decomposition temperature of the samples was investigated under nitrogen atmosphere. TGA curves of chitosan, chi/MMT and chi/MMT/HAP are shown in FIG. 8. From the graph, it can be seen that these materials undergo two steps of degradation. The first step is in the range 100-150° C., due to the loss of solvent molecules. The samples start decomposing from the next step of degradation between 250-300° C. Two parameters are determined from the TGA plot and are shown in Table 3: the onset point, where 20% weight loss took place, and the percentage residue left at 500° C. The onset point for chitosan sheets is 257° C. and for chi/MMT it has increased to 270° C. But in case of chi/MMT/HAP, it has gone up to 278° C. It can be clearly observed from the TGA plot that the degradation behavior of chitosan and its composites are different. MMT has been found to increase the thermal stability of the polymer/clay nanocomposites. Thus, addition of clay is thought to provide a thermal barrier and hence delay the weight loss. Table 3 also shows that addition of HAP particles further increases the onset temperature. Hence, both the inorganic particles are found to enhance the thermal stability of the chitosan. This can also be evidenced from the final residue yield. Percentage yield of the residue increases with the addition of clay and HAP, which is shown in Table 3. So both inorganic particles exhibit a significant delay in the weight loss. This delay could be due to significant interaction between these inorganic particles and polymer, which changes the degradation mechanism.

TABLE 3
TGA results for chitosan and its composites.

| Parameter | Chi | Chi/MMT | Chi/MMT/HAP |
|---|---|---|---|
| Temperature at 20 wt % Loss (° C.) | 257 | 270 | 278 |
| Residue yield at 500° C. (wt %) | 38 | 41.7 | 56.4 |

### Nanomechanical Property

Load-controlled nanoindentaion tests were conducted on chitosan and its composites. The elastic modulus and hardness values are given in Table 4. It was found that there was a significant increase in the elastic modulus of composites from pristine polymer. Chi/MMT and chi/MMT/HAP composites have elastic modulus value ˜8 GPa and ˜9 GPa, respectively. Elastic modulus value for chi/HAP is also shown in the Table 4. With the addition of HAP, elastic modulus increased from 5 GPa to 7 GPa. Although the changes in modulus value for the chi/HAP composites is less than that of chi/MMT composites, the addition of HAP showed a 40% increase in modulus value, which is a significant improvement in mechanical property. So not only MMT, but also HAP, improves the nanomechanical property. This enhancement in mechanical property appears to be due to the well dispersed nanoparticles in the polymer matrix and strong interfacial interaction among chitosan, MMT and HAP.

TABLE 4
Mechanical properties of the composites.

| | 100 μN | | 1000 μN | | 5000 μN | |
|---|---|---|---|---|---|---|
| Sample | Er (GPa) | H | Er (GPa) | H | Er (GPa) | H |
| Chi | 6.94 ± 0.89 | 0.45 ± 0.07 | 4.92 ± 0.35 | 0.17 ± 0.01 | 5.29 ± 0.13 | 0.16 ± 0.007 |
| Chi/HAP | 7.6 ± 1.12 | 0.38 ± 0.06 | 6.96 ± 0.55 | 0.24 ± 0.01 | 7.02 ± 0.22 | 0.22 ± 0.007 |
| Chi/MMT | 8.49 ± 1.91 | 0.52 ± 0.13 | 8.63 ± 0.98 | 0.31 ± 0.05 | 8.28 ± 0.78 | 0.28 ± 0.04 |
| Chi/MMT/HAP | 9.44 ± 1.83 | 0.47 ± 0.11 | 8.17 ± 0.65 | 0.26 ± 0.02 | 9.15 ± 1.0 | 0.28 ± 0.03 |

### AFM Morphology

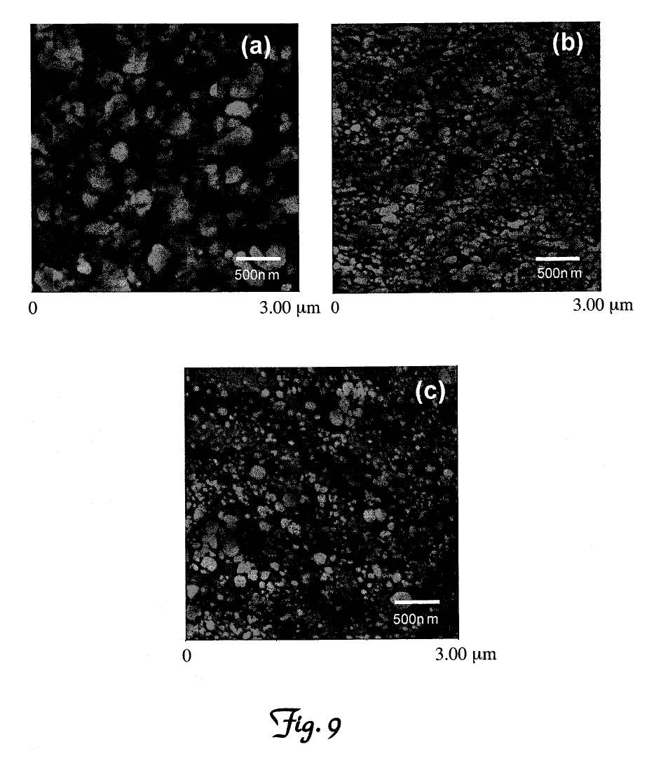

Tapping mode AFM phase images were collected in order to investigate the morphology of the sample. Here, the cantilever oscillates at or slightly below its resonance frequency with amplitude from 20 to 100 nm. The tip slightly taps on the sample surface during scanning. Although this mode of scanning speed is little bit slower, it gives higher lateral resolution. FIGS. 9 (a), (b), and (c) show the tapping mode AFM phase images of chi/MMT, chi/HAP and chi/MMT/HAP composites respectively. In all the images, the darker part is the polymer, and the lighter part is the fillers. The figures exhibit nicely dispersed nanoparticles in the polymer matrix. In chi/MMT composite, clay particles are elongated in shape, having a length of 500 nm and width 200 nm. In the case of chi/HAP, globular HAP particles are homogeneously dispersed in the chitosan matrix, which is clearly visible in the image. The diameter of HAP particles are in the range of 50-100 nm. Comparing both chi/MMT and chi/MMT composites one can distinguish elongated clay particles from the globular HAP particles in chi/MMT/HAP composite. The chi/MMT/HAP image exhibits well distribution of both clay and HAP. This homogeneous distribution of inorganic nanoparticles reveals strong interfacial interaction between nanoparticles and polymer matrix.

### Cell Culture

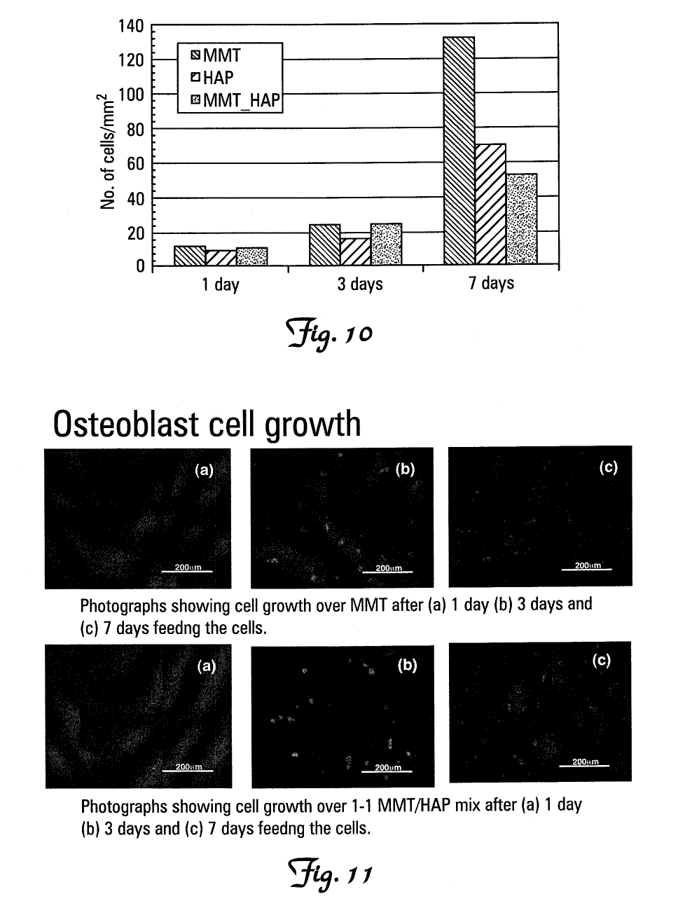

The number of cells grown over each samples after 1 day, 3 days and 7 days were counted from the photographs. The average number of cells per mm² over each sample was calculated and plotted against the number of days for each sample (FIG. 10). From the plot, it is seen that the maximum rate of growth is shown by the cells in MMT. The cells in HAP and MMT/HAP mix also showed a good growth rate. This shows that MMT/HAP mix is a biocompatible material. Also, chitosan is a highly biocompatible natural polymer.

### Materials and Methods

Na-MMT of cationic exchange capacity 76.4 mequiv/100 mg was obtained from Clay Minerals Repository at the University of Missouri, Columbia, Mo., USA. Chitosan of medium molecular weight was purchased from Sigma-Aldrich. Its degree of deacetylation is greater than 85%. Na₂HPO₄, ultrapure bioreagent, glacial acetic acid and NaOH were obtained from J. T Baker. GR grade, CaCl₂ was supplied by EM science. For cell culture experiments, HyQ Dulbecco's Modification of Eagle's Medium (DMEM)-12 (1:1), Fetal Bovine Serum (FBS), G418 solution (antibiotic), and Live-Dead Cell Staining Kit were used. Osteoblast cells (Cell line number CRL-11372) were purchased from American Type Culture Collection (ATCC).

### Preparation of Hydroxyapatite

Hydroxyapatite was prepared by wet precipitation method (Saeria et al., Mater Lett, 57, 4064-4069). 7.2 mM solution of Na₂HPO₄ was made in DI water. In another beaker, 12 mM solution of CaCl₂ was made using DI water. Then 200 ml of CaCl₂ solution was added slowly through a pipette to IL solution of Na₂HPO₄ with continuous stirring. The solution was adjusted to 7.4 pH using NaOH. After mixing the precipitate was allowed to settle down for 24 hours. This precipitate was used to prepare the composites. All the experiments were done at room temperature.

### Preparation of Composites

4.5 gm (90 wt %) of chitosan was dissolved in 225 ml of 1% acetic acid solution. 0.5 gm (10 wt %) of MMT was dispersed in 15 ml of DI water. The MMT suspension was added to the chitosan solution and the solution was continuously stirred for overnight to obtain a good dispersion of clay in chitosan. This solution was then poured into a Petri dish and dried in an oven at 80° C. for 48 hours to form thin sheets. This composite was designated as chi/MMT.

For synthesizing chitosan and hydroxyapatite composites, 40 wt % of freshly prepared HAP precipitate was added to 60 wt % of chitosan solution followed by the same procedure as for chi/MMT composite. This composite was named as chi/HAP.

Similarly, for making chitosan, MMT and HAP composites first 50 wt % of chitosan solution was made. Then 10 wt % of MMT suspension was added to it followed by 40 wt % of HAP precipitate. After this again the same procedure as mentioned earlier was followed to make thin sheets of chi/MMT/HAP composites. Pure chitosan sheets were also made in the same way in order to compare with these composites. All the sheets were characterized using different characterization tools.

### Characterization

### XRD

Wide angle XRD pattern of all the samples were recorded using an X-ray diffractometer, D8 Discover, Bruker AXS Instrument. Cu Kα radiation with a wavelength range of 1.54 Å was used in this experiment. The experiment was done at a scan rate of 2°/min for a scan range of 2θ=2-30°.

### FTIR

Photoacoustic fourier transform infrared (PA-FTIR) spectroscopy spectra were collected using a ThermoNicolet, Nexus, 870 spectrometer. This instrument is equipped with a MTEC model 300 photoacoustic accessory. Continuous mode PA-FTIR spectra were collected in the energy range of 400-4000 cm⁻¹ with a mirror velocity 0.158 cm/s. 1000 scans were collected for each sample at a resolution of 4 cm⁻¹.

### Nanoindentation

Nanoindentation tests were conducted using a Triboscope nanomechanical testing instrument (Hysitron, Mn, USA) at room temperature and pressure. A diamond Berkovich tip was used for the indentation tests. 100 μN, 1000 μN, and 5000 μN loads were applied during the testing. The load controlled indentations were done using a 5-5-5 trapezoidal load function. Tapping mode phase imaging was performed in a multimode AFM having a Nanoscope-IIIa controller equipped with J-type piezo scanner (Veeco Metrology) Santa Barbara, Calif.

### TGA

Thermogravimetric analysis (TGA) was done in a thermogravimetric analyzer, TA Instruments TGA Q500. Samples were placed in platinum pans and heated in nitrogen atmosphere from 25 to 500° C. at a heating rate of 20°/min. The decomposition temperature was calculated using TA universal analysis software.

### Cell Culture

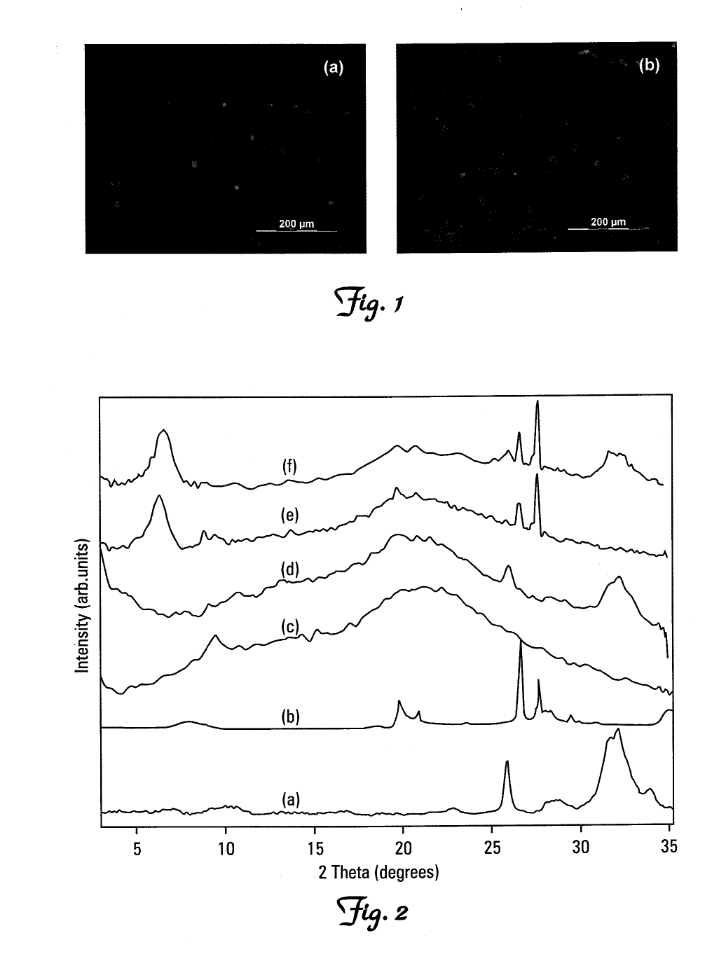

Osteoblasts cells were grown in petri dishes over samples of MMT, HAP and 1:1 MMT/HAP mix. Before feeding the cells, the samples were sterilized under UV light for 1 hour. The initial cell density was 30,000 cells/ml on each sample. The cells were allowed to grow in presence of cell culture medium. 5 ml of the cell culture medium which consisted of 90% HyQ DMEM-12 (1:1), 10% FBS and 0.6% G418 solution (antibiotic) was supplied to each sample. All the samples were placed in an incubator at a temperature of 35° C. The growth of cells over each sample was investigated by taking photographs at a magnification of 200×, after 1 day, 3 days and 7 days of feeding the cells on the sample. Before taking photographs, the samples were stained for the cells to appear fluorescent. Staining was done using Live-Dead Cell Staining Kit which includes: Staining Buffer, Solution A (1 mM Live-Dye) and Solution B (2.5 mg/ml PI). 1 ml of the staining solution, taken from a solution of 1 ml of staining buffer, 1 μl of Solution A and 1 μl of Solution B, was poured on each petridish. The cells were allowed to get stained for 15 minutes. 10 photographs were taken at different locations for each sample at a magnification of 200×. Photographs of the cells grown over MMT/HAP mix after 3 and 7 days are shown in FIGS. 1 (a) and (b) respectively.

Certain embodiments are also described in Katti et al., Biomedical Materials, 3, 034122, 12 pages (2008), which document is incorporated by reference.

### Example 2

### Use of Amino Acids in Clay

Use of nanoclays as structural elements for stiffening scaffolds is new. Three candidates for studies on intercalation of clays with amino acids have been identified (see Table 1; 2-aminopimelic acid, 5-aminovaleric acid or DL-2-aminocaprylic acid). Studies have been performed using 2-aminopimelic acid (C₇H₁₃NO₄). Thus, described herein are the results of studies on clay-amino acid using aminopimelic acid as the amino acid.

In this work, organically modified montmorillonite (OMMT) containing 38% by mass ±2-aminopimelic acid was prepared. For the synthesis of OMMT, 5 g of 45 μm (#325 sieve)≧99% concentration Na-Montmorillonite was heated overnight at 60° C. and then dispersed very slowly in 100 mL deionized (DI) water preheated at 60° C. (solution 1). In another container, 1.9 g≧97% concentration ±2-aminopimelic acid was dissolved in 400 mL DI water preheated at 60° C. (solution 2). The pH of solution 2 was maintained at 1.8 by adding 0.1N HCl. Solution 1 was poured into solution 2 at 60° C. under continuous stirring while maintaining the pH at 1.8 using 0.1N HCl. The solution was stirred vigorously for 1 hour and then allowed to settle. The OMMT was washed with DI water several times until the Cl⁻ was removed from the OMMT. The filtrate was titrated with 0.1N AgNO₃ until no white AgCl precipitate was formed. When no precipitate formed, the OMMT was filtered and centrifuged followed by heating at 80° C. for 24 hours. Further, the OMMT was passed through a 45 μm (#325) sieve.

A Nicolet 850 FTIR spectrometer with KBr beam splitter was utilized in the range of 4000-400 cm⁻¹ with a spectral resolution of 4 cm⁻¹. The FTIR analysis was performed using the transmission technique. IR transparent silicon plates were used as the widow material. Samples were prepared by sandwiching a thin layer of montmorillonite (MMT) or OMMT between two silicon windows. Background spectrum was collected using clean two silicon windows.

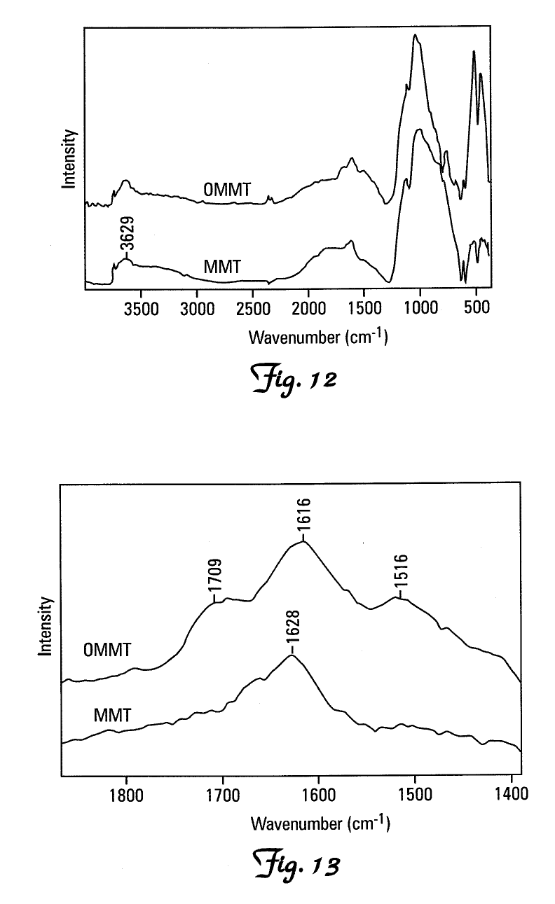

XRD patterns were collected using an X-ray diffractometer, model Philips X'pert, Almelo, Netherlands, equipped with secondary monochromator and Cu-tube using CuKα radiation of wavelength 1.54056 Å. The scanning rate was 20 degree per minute and the scan range was from 2° to 30°. In order to perform XRD analysis, samples were prepared by mounting the MMT or OMMT in an aluminum mount. The transmission FTIR spectra of pure MMT and OMMT are shown in FIG. 12 and the H—O—H bending region of these two spectra is shown in FIG. 13.

Bands at 1628, 1616 cm⁻¹ were attributed to H—O—H bending vibration of interlayer water. The band at 1709 cm⁻¹ was attributed to the disassociated C═O stretching and the band at 1516 cm⁻¹ was attributed to N—H stretching of amino acid. As it can be seen, the H—O—H bending band of interlayer water in OMMT shifts to lower energy by about 12 cm⁻¹. This is thought to be due to the formation of new hydrogen bonds between water molecules and the amino acid molecules that enter into the interlayer. Larger amino acid molecules that are hydrogen bonded to the interlayer water molecules weaken the bending vibration of the interlayer water molecules. In addition, two new bands in OMMT were observed. One occurs at around 1709 cm⁻¹, and this band is attributed to disassociated C═O stretching of carboxylic group. Normally, in amino acids this band occurs at around 1735 cm⁻¹. Due to the disassociation of carboxylic group, this band shifts to lower energy. The other new band appears at around 1516 12 cm⁻¹, and this band is attributed to N—H stretching of amino acid.

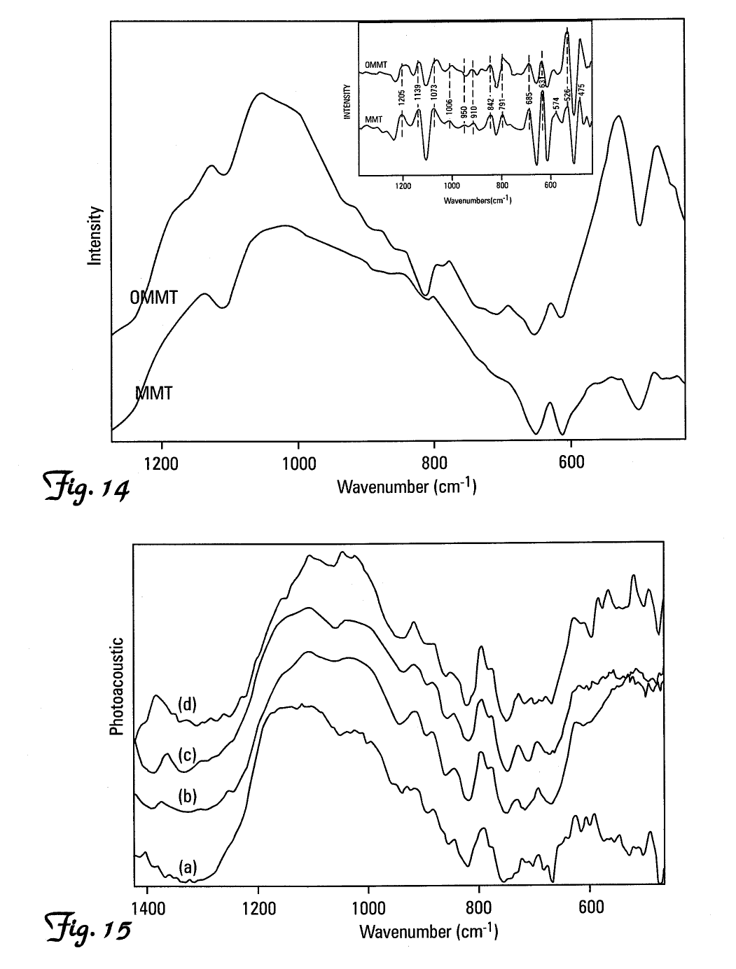

The Si—O stretching region of MMT is shown in FIG. 14. Although C—N band that occurs at around 1100-1050 cm⁻¹ is one of the characteristic bands of amino acids, this band is not observed in OMMT spectrum due to the overlapping of strong Si—O stretching bands of MMT in this region.

Although relative intensities of some Si—O stretching bands of OMMT are changed, no significant position change in the Si—O stretching bands was observed in the OMMT spectrum compared to the MMT spectrum.

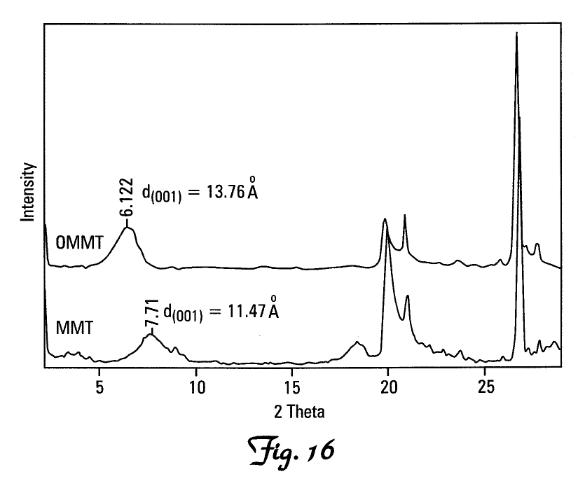

FIG. 15 shows the Si—O region of the FTIR spectra. It was concluded that the organic modifier that influences the MMT structure the most alters the Si—O stretching of MMT the least. While not intended to be limited to any theory, it is believed that the initial pure clay structure was already influenced by the highly polar water molecules that are present in the interlayer, which results in the significantly altered Si—O stretching vibration bands in the FTIR spectrum of MMT. After the clay is modified, some of these water molecules are replaced by the organic modifier resulting unchanged Si—O stretching region in the FTIR spectrum of OMMT. Therefore, it is thought, it is the same reason why significant difference is not seen in the Si—O stretching region of OMMT in this study. XRD results are shown FIG. 16. The d₍₀₀₁₎ spacing of MMT has increased by 2.29 Å after MMT modified by aminopimelic acid, confirming the intercalation of aminopimelic acid in the clay structure.

Thus, clay has been successfully intercalated with an amino acid. This intercalation is demonstrated, e.g., using XRD. This modified clay can be used, e.g., in biopolymer-clay-hydroxyapatite nanocomposite scaffolds for bone tissue engineering. For example, the clay can be incorporated with hydroxyapatite into a polymer solution using a sonicator. A composite of this formulation can then be prepared using freeze drying, or electrospinning fibers.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A composite comprising chitosan, montmorillonite, and hydroxyapatite.

2. The composite of claim 1, wherein the composite further comprises osteoblast cells.

3. The composite of claim 1, wherein the composite further comprises calcium mineralization.

4. The composite of claim 1, wherein the montmorillonite is organically modified montmorillonite.

5. The composite of claim 1, wherein the montmorillonite is unmodified montmorillonite.

6. The composite of claim 1, wherein the composite comprises 2-aminopimelic acid, 5-aminovaleric acid or DL-2-aminocaprylic acid.

7. The composite of claim 1, wherein the composite comprises 2-aminopimelic acid.

8. A method for preparing a composite that comprises chitosan, montmorillonite, and hydroxyapatite, comprising dispersing montmorillonite in a chitosan solution and adding hydroxyapatite precipitate to the solution so as to prepare a composite that comprises chitosan, montmorillonite, and hydroxyapatite.

9. The method of claim 8, wherein the montmorillonite is organically modified montmorillonite.

10. The method of claim 8, wherein the montmorillonite is unmodified montmorillonite.

11. The method of claim 8, wherein the montmorillonite comprises 2-aminopimelic acid, 5-aminovaleric acid or DL-2-aminocaprylic acid.

12. The method of claim 8, wherein the montmorillonite comprises 2-aminopimelic acid.

13. A composite that comprises chitosan, montmorillonite, and hydroxyapatite prepared according to the method of claim 8.

14. The composite of claim 13, wherein the composite further comprises osteoblast cells.

15. The composite of claim 13, wherein the composite further comprises calcium mineralization.

16. A method for treating a patient having a damaged bone, comprising inserting into the patient the composite of claim 1 so as to treat the damaged bone.

17. The method of claim 16, wherein the bone was damaged by an injury.

18. The method of claim 16, wherein the bone was damaged by a disease.

19. The method of claim 17, wherein the damaged bone is a portion of a joint.

20. The method of claim 18, wherein the damaged bone is a portion of a joint.
